(19) United States
(12) Patent Application Publication
(10) Pub. No.: US 2015/0192533 A1
(43) Pub. Date: Jul. 9, 2015

(54) METHOD OF MODELING CONCENTRATION OF REDUCIBLE MOBILE IONIC DOPANT IN SEMICONDUCTOR DEVICE SIMULATOR

(71) Applicant: International Business Machines Corporation, Armonk, NY (US)

(72) Inventors: Mohit Bajaj, Bangalore (IN); Stephen S. Furkay, Burlington, VT (US); Karthik Venkataraman, Bangalore (IN)

(73) Assignee: International Business Machines Corporation, Armonk, NY (US)

(21) Appl. No.: 14/148,174

(22) Filed: Jan. 6, 2014

(51) Int. Cl. G01N 27/04; G01N 33/00

(52) U.S. Cl. CPC G01N 27/04; G01N 33/00; G01N 2033/0095

(57) ABSTRACT

Various embodiments provide systems, computer program products and computer implemented methods. In some embodiments, a system includes a computer-implemented method of determining a dopant concentration in a semiconductor material proximate a metal interface, including determining an electric potential within the semiconductor material at a first voltage range using a known dopant concentration, wherein the dopant is a mobile ion dopant, determining a concentration of a reduced dopant in the semiconductor material, calculating a new expected average dopant concentration for the dopant, calculating a new average dopant concentration for the dopant using the equation with a first damping parameter having a value that is determined by a change in electric potential at a node point in the semiconductor material and determining whether ionic convergence has occurred by determining whether expected dopant concentration deviates from an average concentration by less than a threshold value.

### FIELD

The subject matter disclosed herein relates generally to semiconductors. More particularly, the subject matter disclosed relates to methods of simulating the determination of a mobile dopant concentration in a semiconductor material.

BACKGROUND

Embodiments disclosed relate generally to semiconductor device modeling methods and, more particularly, to the modeling of mobile ionic dopant concentrations in semiconductor devices.

Standard semiconductor device simulators may be used to model behaviors of materials and the molecules found within such materials. Such simulators may provide a physical model based on numerical solutions of coupled drift-diffusion equations (and possibly coupled with hydrodynamic and/or thermodynamic equations) for electrons and ions with appropriate boundary conditions. Semiconductor materials with mobile dopants at ohmic contacts/interfaces have been simulated including their mobile ion distributions, zero-bias potentials, and current-voltage characteristics, for both steady-state bias conditions and for dynamical switching. These simulations are performed to describe physical behaviors in the transport processes responsible for material and molecular behavior in semiconductor films. Numerical methods implemented on device simulators assist in effectively capturing semiconductor device physics and as such, simulation modeling may aid in device design and assist with overcoming manufacturing challenges associated with semiconductor products.

### BRIEF DESCRIPTION

Various aspects of the invention provide for systems, computer program products and computer implemented methods. In some embodiments, a system includes a computer-implemented method of determining a dopant concentration in a semiconductor material proximate an interface of a metal contact and the semiconductor material, the method including determining an electric potential (Ψ) within the semiconductor material at a first voltage range using a known dopant concentration (NDₚᵣₑᵥ), wherein the dopant is a mobile ion dopant, determining a concentration of a reduced dopant (c_{red}) in the semiconductor material, calculating a new expected average dopant concentration (ND_{expnew}) for the dopant, using the equation ND_{expnew}=NDₚᵣₑᵥ−c_{red}, calculating a new average dopant concentration (ND_{new}) for the dopant using the equation ND_{new}=NDₚᵣₑᵥ+a1*(ND_{expnew}−NDₚᵣₑᵥ), wherein a1 is a first damping parameter and determining whether ionic convergence has occurred by determining whether ΔND is below a threshold value, wherein ΔND=max(ND_{new}−ND_{expnew}).

A first aspect provides a computer-implemented method of determining a dopant concentration in a semiconductor material proximate an interface of a metal contact and the semiconductor material, the method comprising: determining an electric potential (Ψ) within the semiconductor material at a first voltage range using a known dopant concentration (NDₚᵣₑᵥ), wherein the dopant is a mobile ion dopant; determining a concentration of a reduced dopant (c_{red}) in the semiconductor material; calculating a new expected average dopant concentration (ND_{expnew}) for the dopant, using the equation ND_{expnew}=NDₚᵣₑᵥ−c_{red}; calculating a new average dopant concentration (ND_{new}) for the dopant using the equation ND_{new}=NDₚᵣₑᵥ+a1*(ND_{expnew}−NDₚᵣₑᵥ), wherein a1 is a first damping parameter having a value that is determined by a change in electric potential at a node point in the semiconductor material; and determining whether ionic convergence has occurred by determining whether ΔND is below a threshold value, wherein ΔND=max(ND_{new}−ND_{expnew}).

A second aspect provides a computer program product comprising program code stored on a computer-readable storage medium, which when executed by at least one computing device, enables the at least one computing device to implement a method of determining a dopant concentration in a semiconductor material proximate an interface of a metal contact and the semiconductor material by performing actions including: determining an electric potential (Ψ) within the semiconductor material at a first voltage range using a known dopant concentration (NDₚᵣₑᵥ), wherein the dopant is a mobile ion dopant; determining a concentration of a reduced dopant (c_{red}) in the semiconductor material; calculating a new expected average dopant concentration (ND_{expnew}) for the dopant, using the equation ND_{expnew}=NDₚᵣₑᵥ−c_{red}; calculating a new average dopant concentration (ND_{new}) for the dopant using the equation ND_{new}=NDₚᵣₑᵥ+a1*(ND_{expnew}−NDₚᵣₑᵥ), wherein a1 is a first damping parameter having a value that is determined by a change in electric potential at a node point in the semiconductor material; and determining whether ionic convergence has occurred by determining whether ΔND is below a threshold value, wherein ΔND=max(ND_{new}−ND_{expnew}).

A third aspect provides a system comprising: at least one computing device configured to determine a dopant concentration in a semiconductor material proximate an interface of a metal contact and the semiconductor material by performing actions including: determining an electric potential (Ψ) within the semiconductor material at a first voltage range using a known dopant concentration (NDₚᵣₑᵥ), wherein the dopant is a mobile ion dopant; determining a concentration of a reduced dopant (c_{red}) in the semiconductor material; calculating a new expected average dopant concentration (ND_{expnew}) for the dopant, using the equation ND_{expnew}=NDₚᵣₑᵥ−c_{red}; calculating a new average dopant concentration (ND_{new}) for the dopant using the equation ND_{new}=NDₚᵣₑᵥ+a1*(ND_{expnew}−NDₚᵣₑᵥ), wherein a1 is a first damping parameter having a value that is determined by a change in electric potential at a node point in the semiconductor material; and determining whether ionic convergence has occurred by determining whether ΔND is below a threshold value, wherein ΔND=max(ND_{new}−ND_{expnew}).

### BRIEF DESCRIPTION OF THE DRAWINGS

These and other features of this invention will be more readily understood from the following detailed description of the various aspects of the invention taken in conjunction with the accompanying drawings that depict various embodiments of the invention, in which:

FIG. 1 shows a lattice structure for illustrating mixed ionic electronic conduction.

FIG. 2 shows a flow diagram illustrating a method according to various embodiments.

FIG. 3 shows a flow diagram illustrating a method according to various embodiments.

FIG. 4 shows an illustrative environment according to various embodiments.

It is noted that the drawings of the invention are not to scale. The drawings are intended to depict only typical aspects of the invention, and therefore should not be considered as limiting the scope of the invention. In the drawings, like numbering represents like elements between the drawings.

### DETAILED DESCRIPTION OF THE INVENTION

The subject matter disclosed herein relates generally to semiconductors. More particularly, the subject matter disclosed relates to methods of simulating the determination of a mobile dopant concentration in a semiconductor material.

As differentiated from conventional attempts at modeling dopant concentrations, various embodiments described herein allow for modeling of concentrations of dopants, holes and electrons using local electric potential. That is, electric potential at at least one grid point in a semiconductor material mesh structure may be used in novel algorithms for modeling concentrations of dopants, electrons and holes according to some embodiments.

According to various aspects described herein, methods for modeling mobile ionic dopant concentrations in semiconductor media are disclosed. Such methods may employ simulation of physical effects which may include electro-reduction of the ionic species at the interfaces of the semiconductor material and metal contacts. These methods, according to embodiments, extend modeling capabilities to both ohmic and Schottky interfaces, and thus embodiments may be applicable to multiple technological applications covering phase change memory (PCM), access diodes for PCM and magnetic random access memories (MRAMs), Resistive RAM, conductive bridging RAM (CBRAM) and other types of memories and other devices. Various embodiments effectively couple conventional semiconductor device simulation with physics-based simulation covering motion of multiple mobile ions, that is, embodiments capture the physics of mixed ionic electronic semiconductors and the physics of electro-reduction kinetics at interface between semiconductor materials and metal contacts.

Embodiments include unique numerical implementation methods for effectively capturing the device physics and as such, embodiments may aid in device design and assist with overcoming manufacturing challenges associated with semiconductor products.

Some embodiments described herein provide methods of modeling transport of mobile ions in a channel of a semiconductor medium using a unique numerical approach using a representation of the concentration of the ion species by quasi-chemical potential (for neutral dopants) and electro-chemical potential (for ionized dopants). Such concentrations are described be the following

$$[ {Cu}_{I} ] = {N}^{′ ( \frac{- q}{{k}_{B} T} ( \frac{{E}_{FD}}{2} + {φ}_{I} )}$$

$$and \\ [ {V}_{Cu} ] = {N}^{( \frac{- q}{{k}_{B} T} ( \frac{{E}_{FD}}{2} - {φ}_{V} )} ,$$

where φ_{I} is a chemical potential at interstitial sites, φ_{V} is a chemical potential at vacancies and the term E_{FD} represents Frenkel pair formation energy. Next, the following equations describe ionic concentrations and current densities associated with such ions.

[Cu_{I}⁺]=[Cu_{I}].f_{I}(Ψ,Φ_{fn},ΔE_{I}) J_{Cu}_{_{I}}_{⁺}−qD_{I}∀[Cu_{I}⁺]−qμ_{I}[Cu_{I}⁺]∀ψ

[V_{Cu}⁻]=[V_{Cu}].f_{V}(Ψ,Φ_{fp},ΔE_{V}) J_{V}_{_{Cu}}_{^{−}}qD_{V}∀[V_{Cu}⁻]−qμ_{V}[V_{Cu}⁻]∀ψ

In the above equations, [Cu_{I}⁺] represents a concentration of ionized Cu interstitial, [Cu_{I}] represents the total number of Cu interstitial in the system (both ionized and un-ionized), f stands for Fermi integral, Ψ is the electrostatic potential, φ_{fn} is the quasi-Fermi level of electrons, φ_{fp} is the quasi-Fermi level of holes, ΔE_{I} represents a difference between the conduction band edge of the semiconductor and the interstitial electronic state within the bandgap, ΔE_{V} represents a difference between the conduction band edge of the semiconductor and the vacancy electronic state within the bandgap. Next, J_{Cu}⁺ represents the current density of the ionized interstitial, J_{VCu}⁻ represents the current density of the ionized vacancy. The J term is a sum of drift term (gradient of potential) and diffusion term (gradient of concentration) with D & μ being the diffusivity and mobility of the ionized species, respectively. By imposing an upper limit on the maximum interface dopant concentration for systems with high doping, for example by numerically defining an upper limit on local driving force for ionic motion as defined by:

∀Φ_{I}⁺=(∀Φ_{I}−f_{I}∀Ψ−(1−f_{I})∀Φ_{fn}) If |∀Ψ_{D}|>E (V/cm)

∀Φ_{V}⁻=(∀Φ_{V}−f_{V}∀Ψ−(1−f_{V})∀Φ_{fp}) ∀Ψ_{D}=0

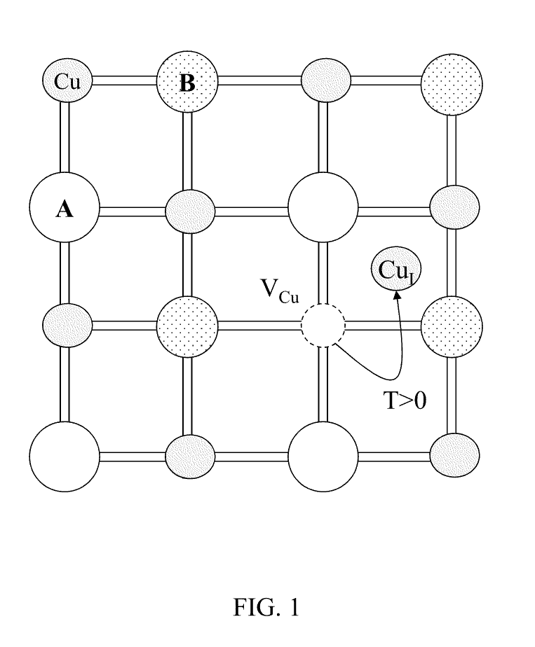

Here, φ_{I}⁺ represents the electrochemical potential of the ionized interstitial defect, φ_{V}⁻ represents the electrochemical potential of the ionized vacancy defect. The equation is derived from above equations. The gradient of Ψ may be set to zero in order to define an upper limit on local driving force for ionic motion, where the gradient of Ψ is the driving force. FIG. 1 shows a lattice structure for illustrating mixed ionic electronic conduction (MIEC). FIG. 1 shows a conventional MIEC ternary compound illustrating a case where the concentration of interstitial dopant atoms (CU_{I},), equals the concentration of lattice structure vacancies (V_{cu}). In this illustration, the dopant is copper. The vacancy, illustrated in dotted lines is a lattice junction, formerly occupied by the interstitially-located copper atom, Cu_{I}, shown by the arrow. At temperature T=0, absolute zero, there would be no defects in the crystal, i.e. [Cu_{I}]=[V_{Cu}]=0. However, at finite temperature T,

$$[ {Cu}_{I} ] = {N}^{′ ( \frac{- {E}_{FD}}{2 {k}_{B} T} )}$$

$$and \\ [ {V}_{Cu} ] = {N}^{( \frac{- {E}_{FD}}{2 {k}_{B} T} )}$$

where N′=total number of possible interstitial sites per unit volume, N=total number of copper lattice sites per unit volume, based on unit cell size under the CGS (centimeter/gram/second) system of N˜N′˜10²²/cc and E_{FD} is energy of defect formation and equals 1.6 eV (assumed currently in density functional theory (DFT) input). Total defect concentration

$$nFD = {( \sqrt{{NN}^{′}} )}^{( \frac{{E}_{FD}}{2 {k}_{B} T} )} ≅ \frac{{10}^{8}}{cc} .$$

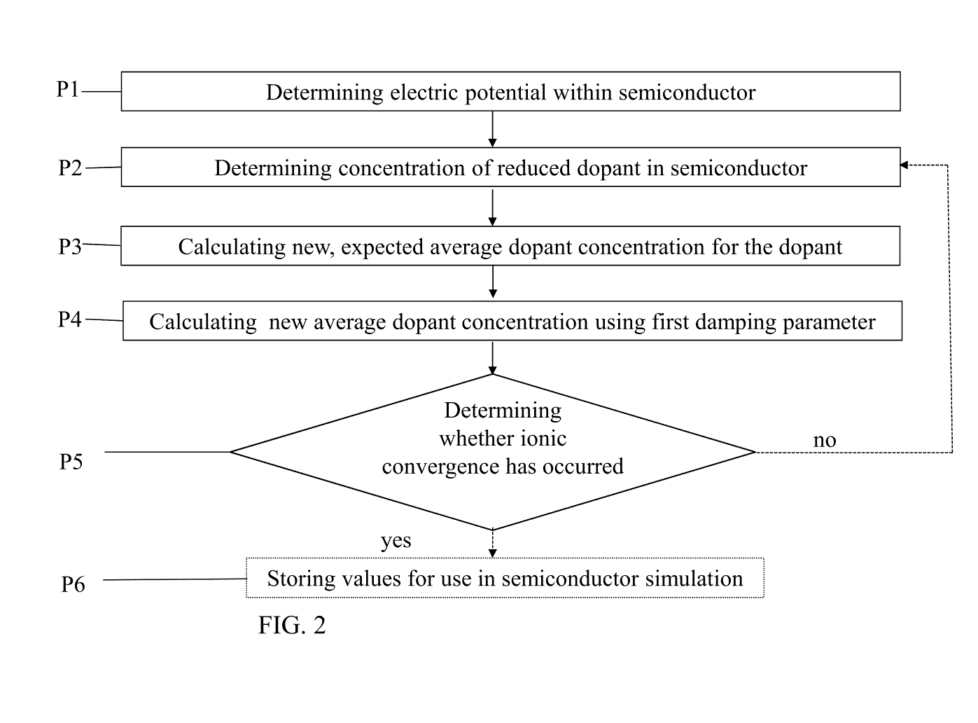

Referring now to FIG. 2, processes in a method for determining a dopant concentration in a semiconductor material are shown in a flow chart. The dopant concentration may include a concentration of an electron acceptor, or an electron donor. FIG. 2 illustrates process P1, which includes determining an electric potential (Ψ) within the semiconductor material at a first voltage range using a known dopant concentration (NDₚᵣₑᵥ), wherein the dopant is a mobile ion dopant. The electric potential (Ψ) may be determined using a semiconductor device simulator supplied with appropriate inputs. Typical voltage ranges may be any voltage ranges appropriate for semiconductor use, now known or later developed for use in the industry. More particularly, a first voltage range may be on the order of approximately +/−3 to 4V. Process P2 includes determining a concentration of a reduced dopant (c_{red}) in the semiconductor material; the concentration of the reduced dopant may be determined by a semiconductor device simulator supplied with appropriate inputs. Initially, c_{red} is set to a value of zero and subsequently c_{red} is calculated from NDₚᵣₑᵥ & ND_{expnew}. In some embodiments of the invention the reduced dopant may include copper; however any appropriate dopant may be used.

Process P3 includes calculating a new expected average dopant concentration (ND_{expnew}) for the dopant, using the equation ND_{expnew}=NDₚᵣₑᵥ−c_{red}, where NDₚᵣₑᵥ is the known dopant concentration used in process P1 and c_{red} is determined in process P2.

Process P4 includes calculating a new average dopant concentration (ND_{new}) for the dopant using the equation ND_{new}=NDₚᵣₑᵥ+a1*(ND_{expnew}−NDₚᵣₑᵥ), wherein a1 is a first damping parameter having a value that is determined by a change in electric potential at a node point in the semiconductor material. a1 is a unique numerical implementation of a damping parameter a1 used to update a new dopant concentration, where the sign and value of a1 is modulated by probing a change in the electric potential (Ψ) at a node point in the mesh structure of the semiconductor material. The value of a1 is iteratively determined by using the following rules: If the sign of (Ψₙ−Ψₙ₋₁) is the same as the sign of (Ψₙ₋₁−Ψₙ₋₂), (i.e. if both are positive values or if both are negative values), then a1=a1ₙ₋₁ multiplied by a Multiplier, else a1=a1ₙ₋₁ divided by a divider, and the Multiplier and the Divider must each be greater than 1. The original value of a1 is may be on the order of approximately 10⁻⁴ to −10⁻³, the damping value is used to assist in bringing the model to convergence, and to prevent ever-growing oscillations of calculated concentration values.

P5 includes determining whether ionic convergence has occurred by determining whether ΔND is below a threshold value, wherein ΔND=max(ND_{new}−ND_{expnew}). As stated above the damping parameter is used to assist in reaching convergence, which may be defined by the value of ΔND being below the threshold value. The threshold value for ΔND may be on the order of approximately a 1% change.

In a case where ΔND is not below the threshold value, i.e., when ionic convergence has not occurred, processes P1-5 may be iteratively repeated, as indicated in FIG. 1, however during reiterations, updated values may be used. Specifically, iterative steps may include, for reiterated process P1, determining an updated concentration of the reduced dopant (c_{rednew}), which may be calculated by determining the updated concentration of c_{rednew} using the non-limiting, illustrative equation: c_{rednew}=c_{red}^{(-q(-E}^{_{defect}}^{-V}^{ₒ}^{-Ψ}^{ₙ}^{)/kT)}, wherein q is a value for electrical charge, in coulombs, E_{defect} is a defect formation energy, Vₒ is a standard reduction potential of the reducing ion species (i.e., the reduced dopant), Ψₙ is a vale for drop in quasi-Fermi level for an electron at a reverse bias metal-semiconductor interface within an atomic distance, (mathematically, this distance may vary between around 5 angstroms to 1 nm), k is Boltzmann's constant, and T is a temperature of the semiconductor material.

Reiterated process P2 includes recalculating the new expected average dopant concentration (ND) for the dopant, using the equation: ND_{expnew}=ND −c_{rednew}. Reiterated processes P3-P5 are the same as described above, and use updated values. Such reiterated steps may be repeated until ionic convergence is determined to have occurred in process P5 (i.e. by determining whether ΔND is below a threshold value, as discussed above with respect to process P5). Once ionic convergence is determined to have occurred, process P6 may be performed. Process P6 includes storing ND_{new} in response to a determination that ionic convergence has occurred. Other values may be stored in process P6, including electron and hole concentrations, along with the potential (Ψ). The values stored in process P6 may be used in further calculation of dopant concentration or in other semiconductor simulator processes.

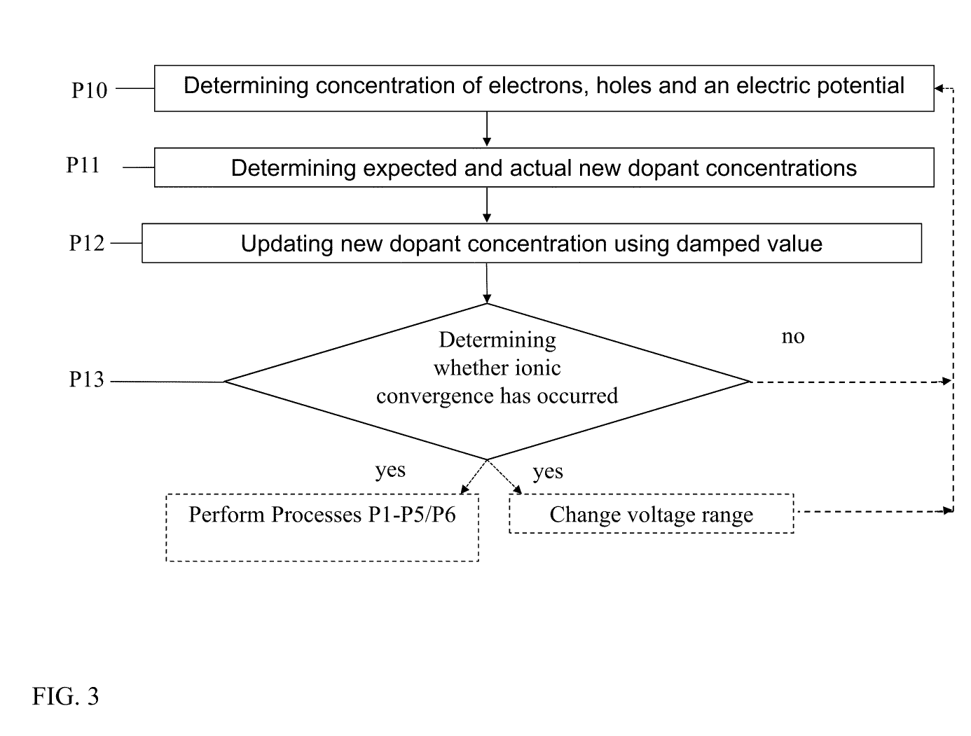

Referring now to FIG. 3, processes which may be performed prior to process P1 are illustrated. Process P10 includes: prior to determining c_{red}, determining a concentration of electrons (n), a concentration of holes (p) and an electric potential (Ψ) within a material of the semiconductor at a first voltage range using the known dopant concentration (NDₚᵣₑᵥ). Electron and hole concentrations and electric potential may be determined using semiconductor simulation software, or any other process now known or later developed. After performing process P10, process P11 may be performed. Process P11 includes determining an expected new dopant concentration (ND_{new}), and an actual new dopant concentration (NDₙₑₓₜ) for the mobile ion dopant, using n, p and Ψ. The determination of such concentrations may be performed using semiconductor simulation software, or any other process now known or later developed. Such determinations may be performed using at least one Poisson equation coupled with a drift-diffusion equation (a drift diffusion equation may be a carrier continuity equation). Other possible methods of determining values for n, p and Ψ may include the use of at least one Poisson equation coupled with one or both of: a thermal solution using lattice heating and a carrier heating solution using carrier heating. The thermal solution and the carrier heating solution may be determined using semiconductor simulation software, or any other process now known or later developed.

Process P12 includes updating NDₙₑₓₜ using a damped NDₙₑₓₜ value in response to a determination that ND_{new} diverges from NDₚᵣₑᵥ by more than a threshold amount. The damped value of NDₙₑₓₜ may be determined using an equation which includes a second damping parameter (a2), for example, NDₙₑₓₜ=NDₚᵣₑᵥ+a2(ND_{new}−NDₚᵣₑᵥ), where a2 has a value based on a change in electric potential before and after a reduction step at a node point in the semiconductor material. Process P13 includes determining whether ionic convergence has occurred by determining whether Δn, Δp, ΔΨ and ΔND are within threshold values. Δn, Δp, ΔΨ and ΔND are: changes in electron concentration, hole concentration, electric potential and dopant concentration. Each delta value is calculated by subtracting previous values from updated values for each parameter. Updated values may be determined using semiconductor modeling software or any appropriate means. Process P13 further includes, in response to Δn, Δp, ΔΨ and ΔND not being within threshold values, iteratively repeating: process P10, using NDₙₑₓₜ in place of ND_{new} and processes P11-13. In response to determining that ionic convergence has occurred, processes P1-P5/P6 may be performed. Also, in response to a determination that ionic convergence has occurred, processes P10-P13 may be repeated at a second voltage range and using NDₚᵣₑᵥ as the starting dopant concentration.

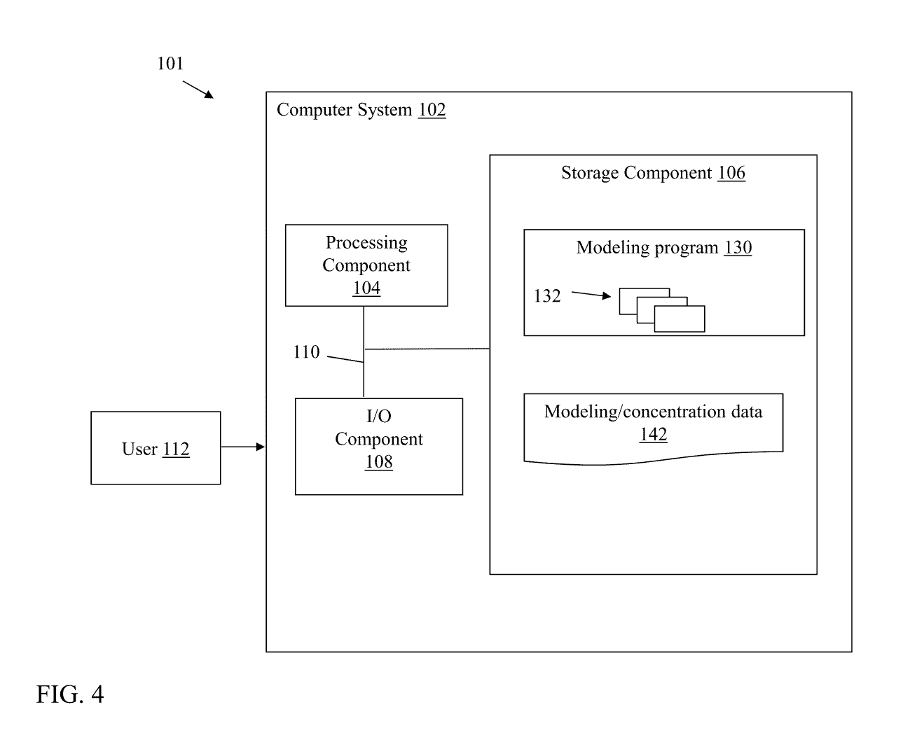

FIG. 4 depicts an illustrative environment 101 for semiconductor modeling. To this extent, the environment 101 includes a computer system 102 that can perform a process described herein in order to model semiconductor materials and dopant concentration. In particular, the computer system 102 is shown as including a modeling program 130, which makes computer system 102 operable to handle modeling of semiconductor materials by performing any/all of the processes described herein and implementing any/all of the embodiments described herein.

The computer system 102 is shown including a processing component 104 (e.g., one or more processors), a storage component 106 (e.g., a storage hierarchy), an input/output (I/O) component 108 (e.g., one or more I/O interfaces and/or devices), and a communications pathway 110. In general, the processing component 104 executes program code, such as the modeling program 130, which is at least partially fixed in the storage component 106. While executing program code, the processing component 104 can process data, which can result in reading and/or writing transformed data from/to the storage component 106 and/or the I/O component 108 for further processing. The pathway 110 provides a communications link between each of the components in the computer system 102. The I/O component 108 can comprise one or more human I/O devices, which enable a human user 112 to interact with the computer system 102 and/or one or more communications devices to enable a system user 112 to communicate with the computer system 102 using any type of communications link. To this extent, modeling program 130 can manage a set of interfaces (e.g., graphical user interface(s), application program interface, etc.) that enable human and/or system users 112 to interact with modeling program 130. Further, the modeling program 130 can manage (e.g., store, retrieve, create, manipulate, organize, present, etc.) data, such as modeling/concentration data 142, etc., using any solution.

In any event, the computer system 102 can comprise one or more general purpose computing articles of manufacture (e.g., computing devices) capable of executing program code, such as the modeling program 130, installed thereon. As used herein, it is understood that “program code” means any collection of instructions, in any language, code or notation, that cause a computing device having an information processing capability to perform a particular function either directly or after any combination of the following: (a) conversion to another language, code or notation; (b) reproduction in a different material form; and/or (c) decompression. To this extent, the modeling program 130 can be embodied as any combination of system software and/or application software.

Further, the modeling program 130 can be implemented using a set of modules 132. In this case, a module 132 can enable the computer system 102 to perform a set of tasks used by the modeling program 130, and can be separately developed and/or implemented apart from other portions of the modeling program 130. As used herein, the term “component” means any configuration of hardware, with or without software, which implements the functionality described in conjunction therewith using any solution, while the term “module” means program code that enables the computer system 102 to implement the functionality described in conjunction therewith using any solution. When fixed in a storage component 106 of a computer system 102 that includes a processing component 104, a module is a substantial portion of a component that implements the functionality. Regardless, it is understood that two or more components, modules, and/or systems may share some/all of their respective hardware and/or software. Further, it is understood that some of the functionality discussed herein may not be implemented or additional functionality may be included as part of the computer system 102.

When the computer system 102 comprises multiple computing devices, each computing device may have only a portion of modeling program 130 fixed thereon (e.g., one or more modules 132). However, it is understood that the computer system 102 and modeling program 130 are only representative of various possible equivalent computer systems that may perform a process described herein. To this extent, in other embodiments, the functionality provided by the computer system 102 and modeling program 130 can be at least partially implemented by one or more computing devices that include any combination of general and/or specific purpose hardware with or without program code. In each embodiment, the hardware and program code, if included, can be created using standard engineering and programming techniques, respectively.

Regardless, when the computer system 802 includes multiple computing devices, the computing devices can communicate over any type of communications link. Further, while performing a process described herein, the computer system 102 can communicate with one or more other computer systems using any type of communications link. In either case, the communications link can comprise any combination of various types of wired and/or wireless links; comprise any combination of one or more types of networks; and/or utilize any combination of various types of transmission techniques and protocols.

The computer system 102 can obtain or provide data, such data 142 using any solution. For example, the computer system 102 can generate and/or be used to generate data 142, retrieve data 142, from one or more data stores, receive data 142a, from another system, send data 142 to another system, etc.

While shown and described herein as a method and system for modeling semiconductor material, it is understood that aspects of the invention further provide various alternative embodiments. For example, in one embodiment, the invention provides a computer program fixed in at least one computer-readable medium, which when executed, enables a computer system to perform a methods of modeling semiconductor material. To this extent, the computer-readable medium includes program code, such as computer system 102 (FIG. 4), which implements some or all of a process described herein. It is understood that the term “computer-readable medium” comprises one or more of any type of tangible medium of expression, now known or later developed, from which a copy of the program code can be perceived, reproduced, or otherwise communicated by a computing device. For example, the computer-readable medium can comprise: one or more portable storage articles of manufacture; one or more memory/storage components of a computing device; paper; and/or the like.

In another embodiment, the invention provides a method of providing a copy of program code, which implements some or all of a process described herein. In this case, a computer system can process a copy of program code that implements some or all of a process described herein to generate and transmit, for reception at a second, distinct location, a set of data signals that has one or more of its characteristics set and/or changed in such a manner as to encode a copy of the program code in the set of data signals. Similarly, an embodiment of the invention provides a method of acquiring a copy of program code that implements some or all of a process described herein, which includes a computer system receiving the set of data signals described herein, and translating the set of data signals into a copy of the computer program fixed in at least one computer-readable medium. In either case, the set of data signals can be transmitted/received using any type of communications link.

In still another embodiment, the invention provides a method of modeling semiconductor material, especially of modeling dopant concentration in the semiconductor material. In this case, a computer system, such as computer system 102 (FIG. 4), can be obtained (e.g., created, maintained, made available, etc.) and one or more components for performing a process described herein can be obtained (e.g., created, purchased, used, modified, etc.) and deployed to the computer system. To this extent, the deployment can comprise one or more of: (1) installing program code on a computing device; (2) adding one or more computing and/or I/O devices to the computer system; (3) incorporating and/or modifying the computer system to enable it to perform a process described herein; and/or the like.

It is understood that aspects of the invention can be implemented as part of a business method that performs a process described herein on a subscription, advertising, and/or fee basis. That is, a service provider could offer to model semiconductor materials, especially to model dopant concentration in the semiconductor material, as described herein. In this case, the service provider can manage (e.g., create, maintain, support, etc.) a computer system, such as computer system 102 (FIG. 4), that performs a process described herein for one or more customers. In return, the service provider can receive payment from the customer(s) under a subscription and/or fee agreement, receive payment from the sale of advertising to one or more third parties, and/or the like.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A computer-implemented method of determining a dopant concentration in a semiconductor material proximate an interface of a metal contact and the semiconductor material, the method comprising:
determining an electric potential (Ψ) within the semiconductor material at a first voltage range using a known dopant concentration (NDₚᵣₑᵥ), wherein the dopant is a mobile ion dopant;
determining a concentration of a reduced dopant (c_{red}) in the semiconductor material;
calculating a new expected average dopant concentration (NDe_{xpnew}) for the dopant, using the equation ND_{expnew}=NDₚᵣₑᵥ−c_{red};
calculating a new average dopant concentration (ND_{new}) for the dopant using the equation ND_{new}=NDₚᵣₑᵥ+a1*(ND_{expnew}−NDₚᵣₑᵥ), wherein a1 is a first damping parameter having a value that is determined by a change in electric potential at a node point in the semiconductor material; and
determining whether ionic convergence has occurred by determining whether ΔND is below a threshold value, wherein ΔND=max(ND_{new}−ND_{expnew}).

2. The method of claim 1, further comprising:
in response to ΔND not being below the threshold value, iteratively performing:
determining an updated concentration of the reduced dopant (c_{rednew}),
recalculating the new expected average dopant concentration (ND_{expnew}) for the dopant, using the equation ND_{expnew}=NDₚᵣₑᵥ−c_{rednew};
recalculating the new average dopant concentration (ND_{new}) for the dopant using the equation ND_{new}=NDₚᵣₑᵥ+a1*(ND_{expnew}−NDₚᵣₑᵥ);
redetermining whether ionic convergence has occurred by redetermining whether ΔND is below the threshold value,
wherein ΔND=max(ND_{new}−ND_{expnew}); and
storing ND_{new} in response to a determination that ionic convergence has occurred.

3. The method of claim 2, further comprising:
determining the updated concentration of c_{rednew} using the equation
c_{rednew}=c_{red}̂(−q(−E_{defect}−Vₒ−Ψₙ)/kT),
wherein q is a value of electric charge in coulombs,
E_{defect} is a defect formation energy,
Vₒ is a standard reduction potential of the reduced dopant,
Ψₙ is a value for a drop in quasi-Fermi level of an electron at a reverse bias metal-semiconductor interface within an atomic distance,
k is Boltzmann's constant, and
T is a temperature of the semiconductor material.

4. The method of claim 1, wherein the dopant includes copper.

5. The method of claim 1, wherein the dopant concentration includes at least one of a donor concentration or an acceptor concentration.

6. The method of claim 1, further comprising:
prior to determining c_{red}, determining a concentration of electrons (n), a concentration of holes (p) and an electric potential (Ψ) within a material of the semiconductor at a first voltage range using the known dopant concentration (NDₚᵣₑᵥ);
determining an expected new dopant concentration (ND_{new}), and an actual new dopant concentration (NDₙₑₓₜ) for the mobile ion dopant, using n, p and Ψ;
updating NDₙₑₓₜ using a damped NDₙₑₓₜ value in response to a determination that ND_{new} diverges from NDₚᵣₑᵥ by more than a threshold amount; and
determining whether ionic convergence has occurred by determining whether Δn, Δp, ΔΨ and ΔND are within threshold values and in response to Δn, Δp, ΔΨ and ΔND not being within threshold values, iteratively repeating:
the determining of n, p and Ψ using NDₙₑₓₜ in place of ND_{new},
the determining of ND_{new} and NDₙₑₓₜ,
the updating of NDₙₑₓₜ, and
the determining whether ionic convergence has occurred.

7. The method of claim 6, further comprising:
determining n, p and Ψ using one of a Poisson equation coupled with a drift-diffusion equation, at least one Poisson equation coupled with lattice heating and at least one drift-diffusion equation, at least one Poisson equation coupled with carrier heating and at least one drift-diffusion equation, or at least one Poisson equation coupled with lattice heating, carrier heating and at least one drift-diffusion equation.

8. The method of claim 6, further comprising:
updating NDₙₑₓₜ using the damped NDₙₑₓₜ value in response to the determination that ND_{new} diverges from NDₚᵣₑᵥ by more than the threshold amount, wherein the damped NDₙₑₓₜ value is calculated using a second damping parameter (a2).

9. The method of claim 6, further comprising:
in response to a determination that ionic convergence has occurred, redetermining n, p and Ψ within the material of the semiconductor at a second voltage range using NDₚᵣₑᵥ;
redetermining ND_{new}, NDₙₑₓₜ using redetermined values for n, p and Ψ;
reupdating NDₙₑₓₜ using a second damped NDₙₑₓₜ value in response to a determination that ND_{new} diverges from NDₚᵣₑᵥ by more than a threshold amount; and
redetermining whether ionic convergence has occurred by redetermining whether Δn, Δp, ΔΨ and ΔND are within threshold values and in response to Δn, Δp, ΔΨ and ΔND not being within threshold values, iteratively repeating:
the redetermining of n, p and Ψ using NDₙₑₓₜ in place of ND_{new},
the redetermining of ND_{new} and NDₙₑₓₜ,
the reupdating of ND_{next,} and
the redetermining whether ionic convergence has occurred.

10. A computer program product comprising program code stored on a computer-readable storage medium, which when executed by at least one computing device, enables the at least one computing device to implement a method of determining a dopant concentration in a semiconductor material proximate an interface of a metal contact and the semiconductor material by performing actions including:
determining an electric potential (Ψ) within the semiconductor material at a first voltage range using a known dopant concentration (NDₚᵣₑᵥ), wherein the dopant is a mobile ion dopant;
determining a concentration of a reduced dopant (c_{red}) in the semiconductor material;
calculating a new expected average dopant concentration (ND_{expnew}) for the dopant, using the equation ND_{expnew}=NDₚᵣₑᵥ−c_{red};
calculating a new average dopant concentration (ND_{new}) for the dopant using the equation ND_{new}=NDₚᵣₑᵥ+a1*(ND_{expnew}−NDₚᵣₑᵥ), wherein a1 is a first damping parameter having a value that is determined by a change in electric potential at a node point in the semiconductor material; and
determining whether ionic convergence has occurred by determining whether ΔND is below a threshold value, wherein ΔND=max(ND_{new}−ND_{expnew}).

11. The computer program product of claim 10, which when executed, enables the at least one computing device to implement the method by performing further actions including:
in response to ΔND not being below the threshold value, iteratively performing:
determining an updated concentration of the reduced dopant (c_{rednew}),
recalculating the new expected average dopant concentration (ND_{expnew}) for the dopant, using the equation ND_{expnew}=NDₚᵣₑᵥ−c_{rednew};
recalculating the new average dopant concentration (ND_{new}) for the dopant using the equation ND_{new}=NDₚᵣₑᵥ+a1*(ND_{expnew}−NDₚᵣₑᵥ);
redetermining whether ionic convergence has occurred by redetermining whether ΔND is below the threshold value,
wherein ΔND=max(ND_{new}−ND_{expnew}); and
storing ND_{new} in response to a determination that ionic convergence has occurred.

12. The computer program product of claim 10, which when executed, enables the at least one computing device to implement the method by performing further actions including:
prior to determining c_{red,} determining a concentration of electrons (n), a concentration of holes (p) and an electric potential (Ψ) within a material of the semiconductor at a first voltage range using the known dopant concentration (NDₚᵣₑᵥ);
determining an expected new dopant concentration (ND_{new}), and an actual new dopant concentration (NDₙₑₓₜ) for the mobile ion dopant, using n, p and Ψ;
updating NDₙₑₓₜ using a damped NDₙₑₓₜ value in response to a determination that ND_{new} diverges from NDₚᵣₑᵥ by more than a threshold amount; and
determining whether ionic convergence has occurred by determining whether Δn, Δp, ΔΨ and ΔND are within threshold values and in response to Δn, Δp, ΔΨ and ΔND not being within threshold values, iteratively repeating:
the determining of n, p and Ψ using NDₙₑₓₜ in place of ND_{new},
the determining of ND_{new} and NDₙₑₓₜ,
the updating of NDₙₑₓₜ, and
the determining whether ionic convergence has occurred.

13. The computer program product of claim 12, which when executed, enables the at least one computing device to implement the method by performing further actions including:
updating NDₙₑₓₜ using the damped NDₙₑₓₜ value in response to the determination that ND_{new} diverges from NDₚᵣₑᵥ by more than the threshold amount, wherein the damped NDₙₑₓₜ value is calculated using a second damping parameter (a2).

14. The computer program product of claim 12, which when executed, enables the at least one computing device to implement the method by performing further actions including:
in response to a determination that ionic convergence has occurred, redetermining n, p and Ψ within the material of the semiconductor at a second voltage range using NDprev;
redetermining NDnew, NDₙₑₓₜ using redetermined values for n, p and Ψ;
reupdating NDₙₑₓₜ using a second damped NDₙₑₓₜ value in response to a determination that ND_{new} diverges from NDₚᵣₑᵥ by more than a threshold amount;
redetermining whether ionic convergence has occurred by redetermining whether Δn, Δp, ΔΨ and ΔND are within threshold values and in response to Δn, Δp, ΔΨ and ΔND not being within threshold values, iteratively repeating:
the redetermining of n, p and Ψ using NDₙₑₓₜ in place of ND_{new},
the redetermining of ND_{new} and NDₙₑₓₜ,
the reupdating of NDₙₑₓₜ, and
the redetermining whether ionic convergence has occurred.

15. A system comprising:
at least one computing device configured to determine a dopant concentration in a semiconductor material proximate an interface of a metal contact and the semiconductor material by performing actions including:
determining an electric potential (Ψ) within the semiconductor material at a first voltage range using a known dopant concentration (NDₚᵣₑᵥ), wherein the dopant is a mobile ion dopant;
determining a concentration of a reduced dopant (c_{red}) in the semiconductor material;
calculating a new expected average dopant concentration (ND_{expnew}) for the dopant, using the equation ND_{expnew}=NDₚᵣₑᵥ−c_{red};
calculating a new average dopant concentration (ND_{new}) for the dopant using the equation ND_{new}=NDₚᵣₑᵥ+a1*(ND_{expnew}−NDₚᵣₑᵥ), wherein a1 is a first damping parameter having a value that is determined by a change in electric potential at a node point in the semiconductor material; and
determining whether ionic convergence has occurred by determining whether ΔND is below a threshold value, wherein ΔND=max(ND_{new}−ND_{expnew}).

16. The system of claim 15, wherein the at least one computing device is further configured to perform actions including:
in response to ΔND not being below the threshold value, iteratively performing:
determining an updated concentration of the reduced dopant (c_{rednew}),
recalculating the new expected average dopant concentration (ND_{expnew}) for the dopant, using the equation ND_{expnew}=NDₚᵣₑᵥ−c_{rednew};
recalculating the new average dopant concentration (ND_{new}) for the dopant using the equation ND_{new}=NDₚᵣₑᵥ+a1*(ND_{expnew}−NDₚᵣₑᵥ);
redetermining whether ionic convergence has occurred by redetermining whether ΔND is below the threshold value,
wherein ΔND=max(ND_{new}−ND_{expnew}); and
storing ND_{new} in response to a determination that ionic convergence has occurred.

17. The system of claim 15, wherein the at least one computing device is further configured to perform actions including:
prior to determining c_{red,} determining a concentration of electrons (n), a concentration of holes (p) and an electric potential (Ψ) within a material of the semiconductor at a first voltage range using the known dopant concentration (NDₚᵣₑᵥ);
determining an expected new dopant concentration (ND_{new}), and an actual new dopant concentration (NDₙₑₓₜ) for the mobile ion dopant, using n, p and Ψ;
updating NDₙₑₓₜ using a damped NDₙₑₓₜ value in response to a determination that ND_{new} diverges from NDₚᵣₑᵥ by more than a threshold amount; and
determining whether ionic convergence has occurred by determining whether Δn, Δp, ΔT and ΔND are within threshold values and in response to Δn, Δp, ΔΨ and ΔND not being within threshold values, iteratively repeating:
the determining of n, p and Ψ using NDₙₑₓₜ in place of ND_{new},
the determining of ND_{new} and NDₙₑₓₜ,
the updating of NDₙₑₓₜ, and
the determining whether ionic convergence has occurred.

18. The system of claim 17, wherein the at least one computing device is further configured to perform actions including:
determining n, p and Ψ using one of a Poisson equation coupled with a drift-diffusion equation, at least one Poisson equation coupled with lattice heating and at least one drift-diffusion equation, at least one Poisson equation coupled with carrier heating and at least one drift-diffusion equation, or at least one Poisson equation coupled with lattice heating, carrier heating and at least one drift-diffusion equation.

19. The system of claim 16, wherein the at least one computing device is further configured to perform actions including:
updating NDₙₑₓₜ using the damped NDₙₑₓₜ value in response to the determination that NDn_{ew} diverges from NDₚᵣₑᵥ by more than the threshold amount, wherein the damped NDₙₑₓₜ value is calculated using a second damping parameter (a2).

20. The system of claim 16, wherein the at least one computing device is further configured to perform actions including:
in response to a determination that ionic convergence has occurred, redetermining n, p and Ψ within the material of the semiconductor at a second voltage range using NDₚᵣₑᵥ;
redetermining ND_{new}, NDₙₑₓₜ using redetermined values for n, p and Ψ;
reupdating NDₙₑₓₜ using a second damped NDₙₑₓₜ value in response to a determination that ND_{new} diverges from NDₚᵣₑᵥ by more than a threshold amount;
redetermining whether ionic convergence has occurred by redetermining whether Δn, Δp, ΔΨ and ΔND are within threshold values and in response to Δn, Δp, ΔΨ and ΔND not being within threshold values, iteratively repeating:
the redetermining of n, p and Ψ using NDₙₑₓₜ in place of ND_{new},
the redetermining of ND_{new} and NDₙₑₓₜ,
the reupdating of NDₙₑₓₜ, and
the redetermining whether ionic convergence has occurred.
